(19) United States
(12) Patent Application Publication
(10) Pub. No.: US 2019/0346355 A1
(43) Pub. Date: Nov. 14, 2019

(54) METHOD FOR ESTIMATING IN REAL-TIME STOCKPILE PARTICLE SIZE DISTRIBUTION ASSOCIATED TO A LEVEL-BASED DISCRETIZATION

(71) Applicants: Jack Oliver Adolf LUNDIN, Vancouver (CA); Maria Nathalie RISSO SEPULVEDA, Tucson, AZ (US); Junhyeok PARK, Tucson, AZ (US)

(72) Inventors: Jack Oliver Adolf LUNDIN, Vancouver (CA); Maria Nathalie RISSO SEPULVEDA, Tucson, AZ (US); Junhyeok PARK, Tucson, AZ (US)

(21) Appl. No.: 16/407,931

(22) Filed: May 9, 2019

Related U.S. Application Data
(60) Provisional application No. 62/671,242, filed on May 14, 2018.

(51) Int. Cl. G01N 15/02; G01N 15/10; G01N 33/24; G06T 15/08; G06T 17/20

(52) U.S. Cl. CPC G01N 15/0227; G01N 15/10; G01N 33/24; G06T 2210/56; G06T 17/20; G01N 2015/1087; G06T 15/08

(57) ABSTRACT

A method for performing a real-time estimation of the particle size distribution in the outer surface of a stockpile is provided. The method involves combining information about real-time stockpile volumes, rock parameters, and measures of particle size distribution obtained over a period of time which is long enough to provide consistent operational data points. The method involves receiving information about the stockpile volume and/or enough disperse level measurements to estimate the stockpile volumetric distribution which is later combined with particle size information. The information can be obtained from a sensor or a method which generates either a particle size distribution or size related parameters associated to an estimated distribution. Once all information is gathered, a particle size distribution, along with rock properties in the stockpile are obtained.

This non-provisional application claims the benefit under 35 § 119(e) to U.S. Provisional Application No. 62/671,242, filed on May 14, 2018, all of which are hereby expressly incorporated by reference into the present application.

The invention relates, in general, to a method and system for estimating spatial variation of particle sizes and rock properties in a stockpile, and specifically, to a method and system for providing real-time estimation of particle size and rock properties on the outer surface of a stockpile using three-dimensional (3D) sensor data.

### BACKGROUND

In the solid materials industry, particularly mineral resources, a challenge facing operations is the ability to predict and manage particle size distributions within the coarse ore stockpile. Run-of-mine ore which begins comminution through a crushing circuit is generally stockpiled prior to entering the milling process. Naturally, the stockpile will form a heterogeneous accumulation of coarser particles on the periphery of the pile and finer particles accumulating towards the top and center of the pile. This leads to a segregation phenomenon that affects the efficiency and operability of the comminution cycle such as mineral processing in SAG milling operations. The segregation phenomenon has adverse cost and safety effects, since it requires an additional procedure for blending before the ore is transferred to the mill. Ideally, variations in particle size distribution should be bounded, since they affect energy consumption and efficiency of grinding.

To alleviate the segregation problem, industry practice usually sorts the stockpiles using mobile industrial equipment, such as dozers or excavators. However, this method of utilizing mobile equipment is not fully effective nor safe. In 2016, Mine Safety and Health Administration (MSHA) issued a stockpile accident safety alert due to seven stockpile-involved dozer accidents. The particle size and property prediction/estimation can be used to create proper/safer dozer operational guidelines for stockpile sorting.

Commercially available stockpile sensors provide a 3D representation of a stockpile surface and its deformation by using laser scanning or radar. The geometric model calculates the volume of the stockpile simply from the volume of a mesh object using the 3D representation. While this method helps in monitoring stockpile conditions, it does not have the capability to provide any information on the trajectory of the crushed material, or the problems related to particle segregation.

From a macro point of view, there is a point in the comminution cycle that lacks information to optimize safety and efficiency. Thus, there exists a need for a standardized system that utilizes real-time mathematical modeling to estimate the spatial variation of particle sizes, as well as rock properties throughout stockpile surfaces. An operation capable of utilizing such a standardized system will have the capabilities of optimizing operational conditions of mineral or material processing, reconciling ore, waste, and other bulk materials against production data from other sources, and implementing proper/safer equipment operational guidelines for the stockpile sorting.

### SUMMARY

The invention solves the problems with conventional solutions by providing a new method for representing particle size distribution and particle/rock properties in stockpiles. The invention captures this representation in real-time, which provides valuable information to an operation for aide in important decision making. The value in real-time representation of stockpile information is the potential to improve efficiency and reduce operating costs of the overall comminution and processing phases.

Further advantages of the invention are described in more detailed sections below and include short term mine planning optimization, material reconciliation, effective stockpile sorting guidance, classification of safe equipment operability zones, real-time data analysis, and machine control optimization.

Further scope of applicability of the invention will become apparent from the detailed description given hereinafter. However, it should be understood that the detailed description and specific examples, while indicating embodiments of the invention, are given by way of illustration only, since various changes and modifications within the spirit and scope of the invention will become readily apparent from this detailed description to those skilled in the art.

### BRIEF DESCRIPTION OF THE DRAWINGS

FIG. 1 illustrates a typical stockpile system in a mining operation and segregation problem inside of the stockpile;

FIG. 2A illustrates a level sensor for acquiring stockpile surface profile and volumetric information according to an embodiment of the invention;

FIG. 2B illustrates a volumetric sensor for acquiring stockpile surface profile data and volumetric information according to an embodiment of the invention;

FIG. 3 illustrate sequential steps and their connections in the main algorithm execution according to an embodiment of the invention;

FIGS. 4A and 4B illustrate a 3D voxel grid of the stockpile surface according to an embodiment of the invention;

FIG. 5 illustrates steps associated to the pre-processing component of the main algorithm according to an embodiment of the invention;

FIG. 6 illustrates an example of the execution of the method and associated steps; and

FIG. 7 is a block diagram illustrating an exemplary computing device.

### DETAILED DESCRIPTION

The invention is directed to a method and system for estimating a spatial variation of particle size in a stockpile, and specifically to a method and system for providing real-time estimations of the particle size distribution and rock properties on the outer surface of a stockpile using integrated sensor data.

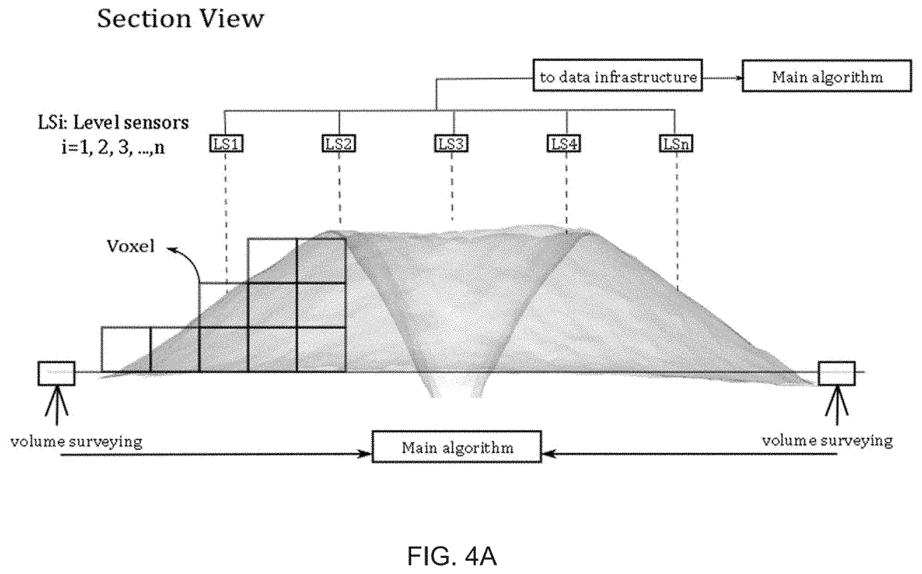

FIG. 3 illustrates a process for estimating particle distribution in a stockpile according to aspects of the present invention. The process begins with the acquisition of stockpile surface information from sensors positioned around/near a stockpile (Step 301). The surface information which may optionally be pre-processed (Step 303) is then used to generate a volumetric estimation and distribution parameters of the stockpile (Steps 305 and 307). A 3D voxel grid of the stockpile is generated, and the particle distribution is estimated by associating a particle size to each voxel in the 3D grid using a representative particle size function (Step 309).

The following assumptions are incorporated into the algorithm/process.

A1: Gravity is one of the main forces affecting the segregation process.

A2: The effects of machinery operating on the stockpile may not initially be considered.

A3: The angle of repose is defined by the material type;

A4: There are sensors that measure the stockpile level;

A5: The sensor data considered to get information on particle size is of adequate resolution, taken with appropriate angles of view and following standardized methods.

A6: Sampling time associated to the measure of assumptions A2-A5 should be appropriated since they will affect precision of the algorithm.

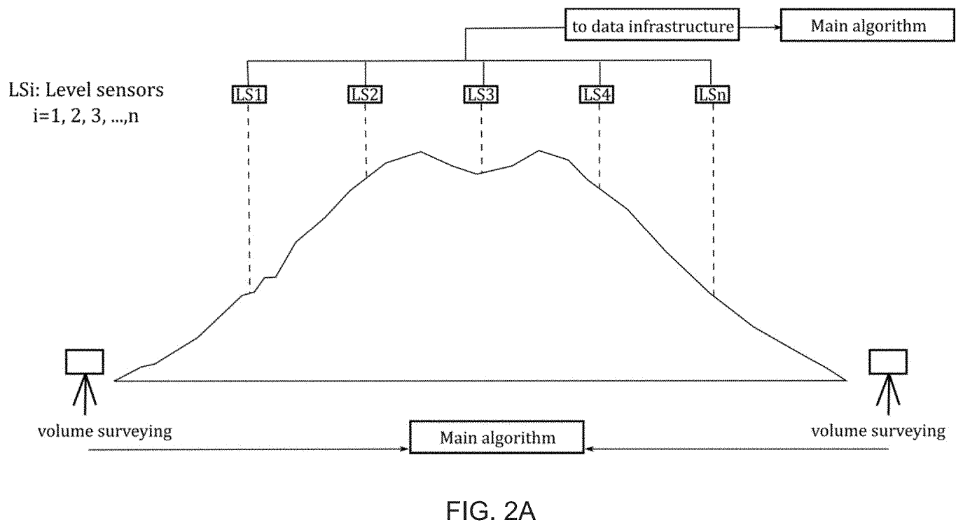

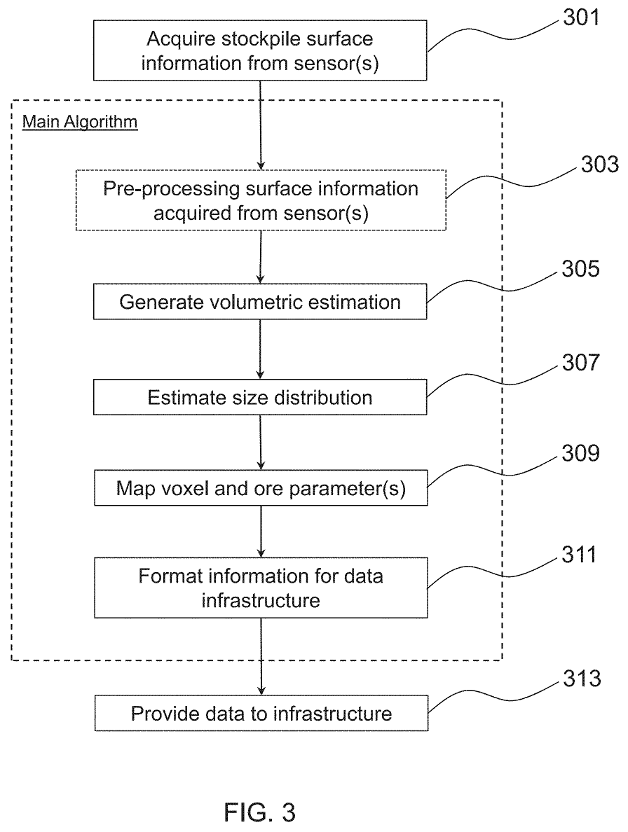

As shown in FIGS. 2A and 2B, stockpile surface information is acquired from sensors positions around/near the stockpile. :A single volumetric sensor, as shown in FIG. 2B may be used, or a plurality of level sensors may be used as shown in FIG. 2A. These sensors can be simple, if they return the level of a particular point in the stockpile or 3D if they return a point cloud. In addition, the sensor data may provide additional properties of the material in the stockpile. In either case the sensor is well calibrated, its location is known, and its accuracy is acceptable for the application.

A characterization of the outer shape, e.g. surface, of a stockpile is generated using the real-time sensor data acquired from a volumetric sensor and/or one or more level sensors (604). This characterization may be a 3D point cloud or a fitted surface depending on the sensor data type. Subsequently, this data is then used to compute an online volume estimation (305).

Starting from a steady state condition, particle analysis is performed to measure over the surface of the stockpile (all sections should be measured). These measurements are periodic, for example, every day or once every several days. As shown in the FIG. 2A, these measurements are acquired from a plurality of level sensors. However, this is merely for illustrative purposes as any sensor that allows generation of an acceptable estimation of particle size, for instance as a statistical distribution, may be used. Examples of sensors include level sensors and/or 3D volumetric sensors.

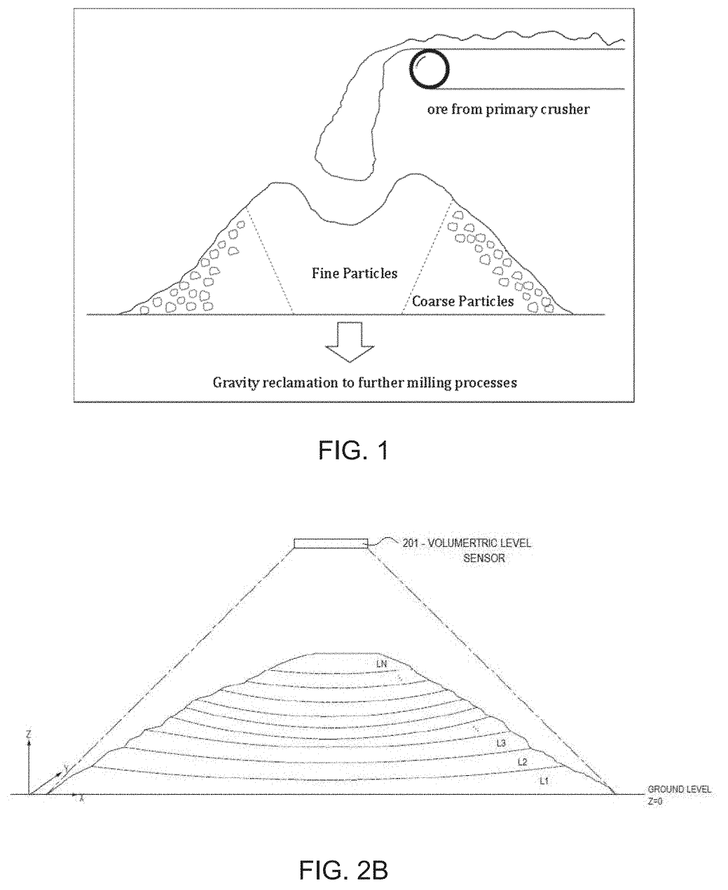

As shown in FIG. 1, due to the nature of the particles in the stockpile, the particles naturally migrate to form sections having similar particle size. As a result, each section of the stockpile represents an estimated unique particle size distribution. The level of resolution depends on the size of the stockpile and sensor data accuracy.

Using historical information from spatial, volumetric, particle size and rock property measurements/estimations, the algorithm fits a function that represents the relationship between discrete levels (defined in terms of ranges of voxels or “zones”) and a size distribution. This is performed using a system identification algorithm, which for instance, can be based on a regressor with variable memory. Because the particle size distribution is bounded, the distributions by level will not have extreme variability.

Due to the bounded nature of the distribution and the conservation of energy in the process, it is possible to prove that the estimation error will remain bounded, and thus the particle size estimated by zone (denoted by R below), will also remain close to the sampled measure obtained periodically.

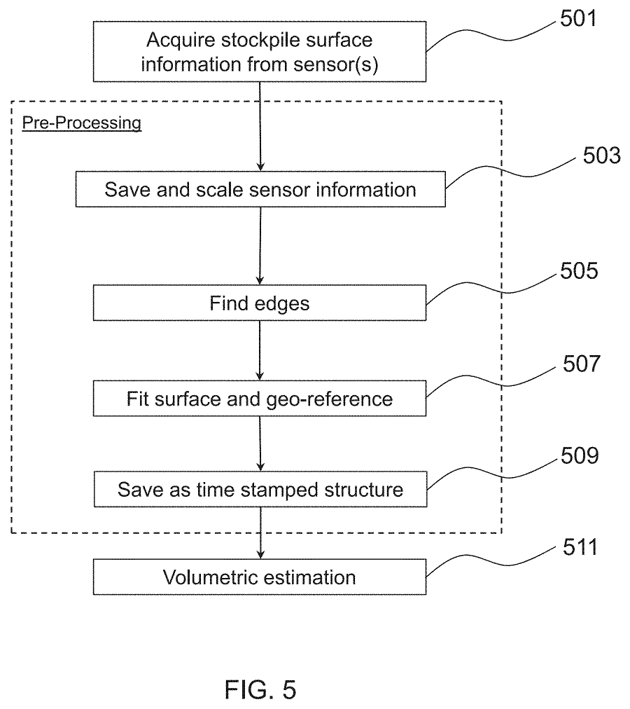

The sensor data may optionally be pre-processed to first obtain the spatial and volumetric information about the stockpile. Then, the processed information is discretized in a 3D model, time stamped and stored as a collection of voxels characterized by a side, d as shown in FIG. 5. Accuracy of this representation will be a function of the sensors accuracy and it can be used for online stockpile volumetric estimation by summing over the voxels in the structure.

A vector V of properties is associated with each voxel. V=[(x, y, z), d, Rᵢ, rp], where (x,y,z,) is the voxel center coordinate, d is the voxel side, Rᵢ is a region which describes constant particle size distribution parameters, for example, R={coarse, medium, fine}, and rp is rock properties, such as hardness, density, ore grade, and the like. The region Ri is defined with respect to spatial coordinates within the stockpile model, and it represents a zone of constant particle size distribution parameters (Step 309).

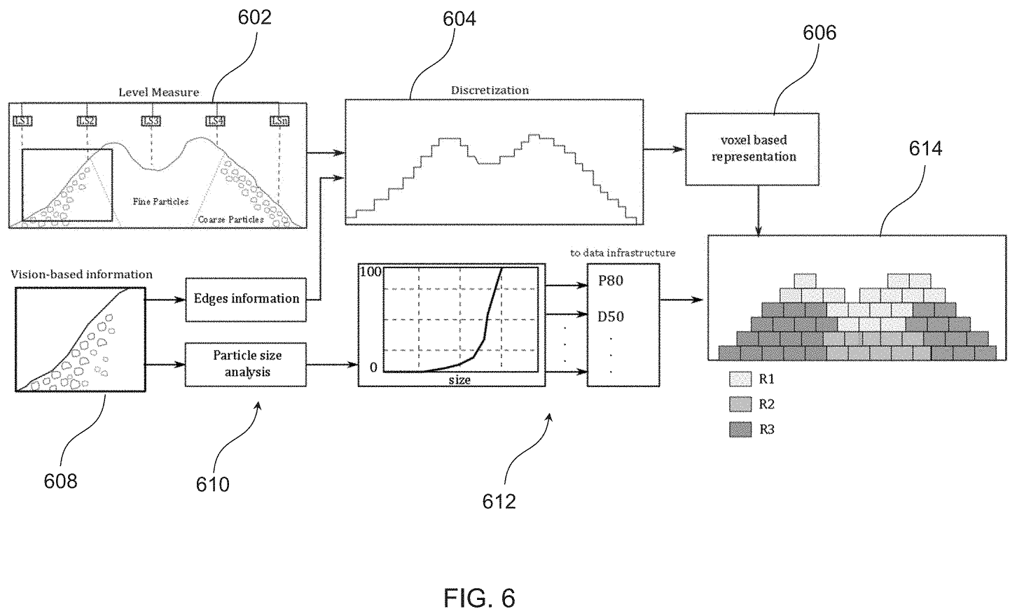

The estimation of size distribution uses information from sensors (cameras, complementary vision-based sensors, lidar, and the like) which cover the whole surface of the stockpile to determine key parameters of particle size distribution functions. Gaudin-Schumann or Rosin-Rammler distributions have been commonly used to represent the statistical distribution of particle sizes of rock fragmentation. Methods for this can include classic image processing, vision-based, spectral sensors, and the like. FIG. 6 illustrates an example using image-based analysis. The particle size estimation may be determined using one or more known algorithms using commercial software, for example SPLIT Online©, Motion Matrics©, WipFrag©, or another computer vision-based technique.

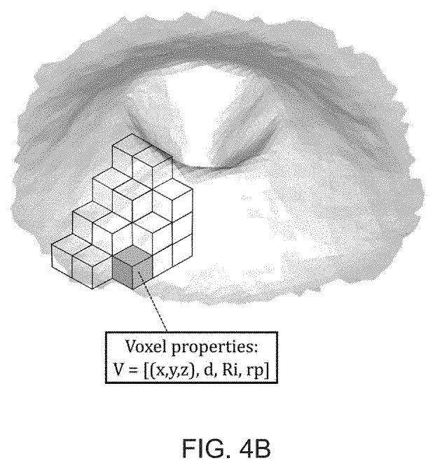

From the collected information, numbered zones are defined as ranges of voxel areas. To each region a representative particle size variable, for example P80, is associated. Thus, the size per zone is estimated as a function of the stockpile spatial representation. The result from this step will be the creation of a map that defines the stockpile zones and the particle size associated to them as shown in FIG. 4B.

If a volumetric sensor is used to acquire the stockpile surface information, the point cloud provided by the volumetric sensor can be used to generate a 3D grid of the stockpile surface as shown in FIG. 2. Discrete volume elements are then defined and used to generate a matrix with the stockpile levels (x,y,z) where x and y are positions in the horizontal plane, and z is the level. With this discretization, the stockpile volume is estimated, in real time, by performing a summation that represents the stockpile volume.

If level sensors are used to acquire the stockpile surface information, they only provide the level of a point in the stockpile. However, using the level sensor coordinates, a point x,y can be associated to each measurement from the acquired images to estimate a contour of the stockpile. The contour, combined with the level measured, can be used to fit a function that better represents the overall shape. This function can then be discretized to obtain the 3D grid, in the same manner as in the case where a volumetric sensor is used. Note that in this case, the more sensors available the better the volume estimation.

With the discretized 3D map of the stockpile, the representative particle size function can be used to associate a particle size to each point in the voxel grid.

The estimated particle distribution can be integrated into the data infrastructure of the applicable operation, propagated over the control network, and may be output via a user interface (Step 311). Moreover, the data may be provided to one or more existing applications in an operation (Step 313). Applications for real-time representation of stockpile information are available within the comminution and processing phase such as mineral processing control centers, short term mine planning, material reconciliation, stockpile sorting, classification of safe equipment operability zones, real-time data analysis, and/or machine control optimization.

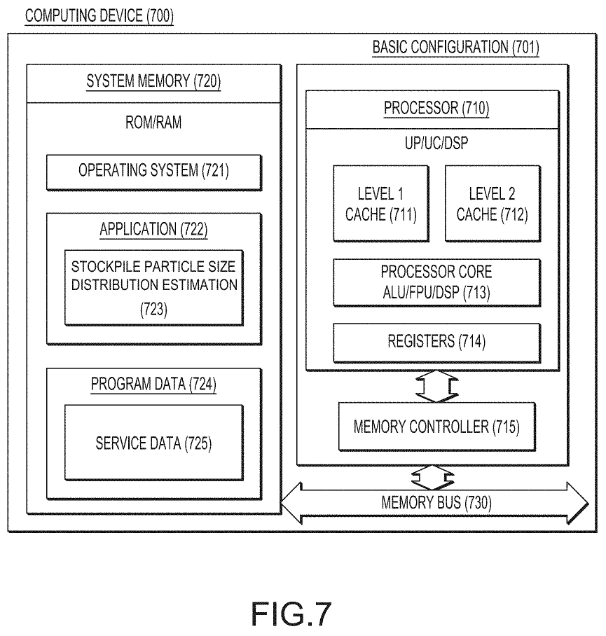

FIG. 7 is a block diagram illustrating an example computing device 700, that is arranged for providing particle distribution in stockpile estimations in accordance with the present disclosure. In a very basic configuration 701, computing device 700 typically includes one or more processors 710 and system memory 720. A memory bus 730 can be used for communicating between the processor 710 and the system memory 720.

Depending on the desired configuration, processor 710 can be of any type including but not limited to a microprocessor (μP), a microcontroller (μC), a digital signal processor (DSP), or any combination thereof. Processor 710 can include one or more levels of caching, such as a level one cache 711 and a level two cache 712, a processor core 713, and registers 714. The processor core 713 can include an arithmetic logic unit (ALU), a floating point unit (FPU), a digital signal processing core (DSP Core), or any combination thereof. A memory controller 715 can also be used with the processor 710, or in some implementations the memory controller 715 can be an internal part of the processor 710.

Depending on the desired configuration, the system memory 720 can be of any type including but not limited to volatile memory (such as RAM), non-volatile memory (such as ROM, flash memory, etc.) or any combination thereof. System memory 720 typically includes an operating system 721, one or more applications 722, and program data 724. This described basic configuration is illustrated in FIG. 7 by those components within dashed line 401.

Computing device 700 can have additional features or functionality, and additional interfaces to facilitate communications between the basic configuration 701 and any required devices and interfaces. System memory 720, removable storage 751 and non-removable storage 752 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 400. Any such computer storage media can be part of device 700.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

Some advantages in utilizing the disclosed embodiments are provided (but not limited to) below.

Real-time analysis: Storage of fragmented material in the stockpile is a point in the comminution cycle that lacks real-time information. The stockpile is a material buffer that works as a security blanket to industrial operations. The volume and characteristics of the stockpile determine how continuously and efficiently an operation can continue. Distribution of rock properties are continuously changing, due to rock types, blasting designs, and stockpile feeding conditions. Understanding the characteristics and trends in real-time provides operators to make decisions that could lead to cost savings, enhanced safety performance, and efficiency improvements, among others.

Processing Facility Operating Performance: SAG mills are very sensitive to the levels and segregation within the crushed ore stockpile. Without understanding the physical properties of the stockpile, eventually bigger rocks find their way through the chute and into the feed line causing high fluctuations in particle size variability. SAG performance can be improved with the least amount of segregation. Real-time characterization of the stockpile is therefor a critical component to reducing segregation and particle segregation variability.

Mine Planning: Stockpiles act as material buffers within the mine operation. An efficiently-ran operation will keep the plant feed stockpile at enough volumes to ensure a continuous flow of adequately sized crushed ore into the processing plant. With the proposed invention, ore tonnage and volume information can be collected and provide indication as to how the short term mine planning team needs to set production targets.

Reconciliation: Stockpiles are often used as indicators for material reconciliation against the production and operations data which is being tracked from the mine operations (blasting, loading and hauling, rehandling, crushing etc.). It is a high priority to understand where all the blasted material is being handled, especially in high-grade precious metal deposits.

Stockpile Sorting and Machinery Guidance: Mobile equipment such as excavators or dozers are often used to manipulate the stockpile shape and rock segregation. Real-time stockpile data can be integrated into equipment control systems and used to guide, man operated and/or autonomous vehicles.

Safety: The upper part of the stockpile can have unstable zones that are vulnerable to the rat hole (zone of the stockpile which ore falls through a chute below). If volumetric sensors are used, or if enough level sensors are provided on top of the stockpile that cover the rat hole, the distribution function can be used to track the movement of the estimated unstable zone boundaries. So, after enough data is collected, estimates of the limits of the safe zone outsize the unstable areas can be provided, thus increasing the mobile equipment operator's safety. These boundaries can also be programmed into autonomous or semi-autonomous machinery for the operations that utilize this technology.

Other applicable areas include: Leach pads, waste dumps, bulk commodity distribution centers (ports, smelters, plants, and the like).

Particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A method for estimating particle distribution in stockpile, comprising:
acquiring sensor data of a stockpile surface;
generating, from the acquired sensor data, volumetric estimation parameters of the stockpile;
generating a three-dimensional (3D) voxel grid; and
estimating a particle distribution in the stockpile by associating a particle size to each voxel in the 3D grid using a representative particle size function.

2. The method of claim 1, wherein the acquired sensor data is surface images acquired using a volumetric sensor.

3. The method of claim 2, wherein the 3D voxel grid is generated from a point cloud provided by the volumetric sensor.

4. The method of claim 1, wherein the sensor data includes surface images acquired using a plurality of level sensors.

5. The method of claim 4, wherein the 3D voxel grid is generated based on contour information derived from the level sensor data.

6. The method of claim 1, wherein the sensor data is acquired at a predefined time interval.

7. A system for estimating a spatial variation of particle size in a stockpile, comprising:
a sensor for acquiring stockpile surface information;
a memory; and
one or more processors configured to
receive surface information of a stockpile from the one or more sensors;
define, from the acquired surface information, a plurality of regions having similar particle size distribution parameters in the stockpile;
generate a 3D voxel grid; and
estimate a particle distribution in the stockpile by associating a representative particle size to each voxel in the 3D voxel grid using a representative particle size function.

8. The system of claim 7, wherein the sensor is a volumetric sensor.

9. The system of claim 8, wherein the 3D voxel grid is generated from a point cloud provided by the volumetric sensor.

10. The system of claim 7, further comprising a plurality of level sensors.

11. The system of claim 10, wherein the 3D voxel grid is generated based on contour information derived from the level sensor data.
